(19) United States
(12) Patent Application Publication
(10) Pub. No.: US 2023/0277014 A1
(43) Pub. Date: Sep. 7, 2023

(54) CONNECTED DISPENSING POINT FOR A CLEANING AND/OR DISINFECTING SOLUTION

(71) Applicant: GELAAS, Versailles (FR)

(72) Inventors: Suat TOPSU, Versailles (FR); Hongyu GUAN, Versailles (FR); Hassan GUANNOUM, Courbevoie (FR)

(21) Appl. No.: 18/006,281

(22) PCT Filed: Jul. 23, 2021
(86) PCT No.: PCT/EP2021/025280

§ 371 Date: Jan. 20, 2023

(30) Foreign Application Priority Data
Jul. 24, 2020 (FR) 2007825

(51) Int. Cl. A47K 5/12; G08C 17/02; A61L 2/24; A61L 2/00

(52) U.S. Cl. CPC A47K 5/1217; G08C 17/02; A61L 2/24; A61L 2/0088; G08C 2201/93; H04W 76/10

(57) ABSTRACT

The invention deals with a dispenser 1 of a cleaning and/or sanitizing solution with a radiofrequency communication module 2B for remote monitoring by a portable communication device UL to deliver a preset dose of the cleaning and/or sanitizing solution. The invention also deals with a control process 6 of such a dispenser 1, wherein the control process 6 includes an identification step 62 of the portable communication device UL by the dispenser 1 and—under condition of authorization after identification—a step of release 63 of the cleaning and/or sanitizing solution.

### TECHNICAL FIELD

The technical context of the present invention is public sanitary devices that make it possible to deliver doses of cleaning and/or sanitizing solutions in order to mitigate the propagation of viruses or bacteria in the population.

More specifically, the invention relates to a connected dispenser of a cleaning and/or sanitizing solution, and to a control process of such a dispenser.

### PREVIOUS STATE OF THE ART

After the advent of large scale sanitary crises and associated pandemic risks, dispensers of hand sanitizing gel have shown up in the public space in order to allow the people wash their hands when moving across the public space. These dispenser are generally shaped as totem including a tank and a dispensing system with a manual command, a foot command, or even sometimes thanks to a presence sensor, such as a proximity sensor—for example an infrared sensor. When moving their hands under hand sanitizing gel dispensing nozzles, a user can monitor the dispensing system to release a preset dose of hand sanitizing gel.

Such dispensers have several drawbacks.

A first drawback is a lack of visibility of known dispensers: if they are not positioned in perfectly visible places, potential users won't see them enough. On the other hand, standing them in clearly visible places may affect the organization of the public space by restricting the movement of people across the space where the dispensers are positioned.

A second drawback is the ergonomics of the known dispensers. Models with a manual or foot triggering handle require pressing the handle to release a dose of hand sanitizing gel. This physical manipulation of the dispenser helps disseminate viruses and bacteria between successive users of the known dispenser, contrarily to the intended goal.

In addition, the known dispensers do not have very sophisticated dispensing systems. Thus, a user may have to trigger the handle several times to get more than a dose, which leads to wasting hand sanitizing gel.

At last, a third known drawback is difficulties to organize an efficient and profitable maintenance and refurnishing service for the known dispensers. Indeed, with no indicator relative to their functioning, known dispensers may remain several days without replenishment or repair after a material breakdown. This leads to too long and too frequent service disruptions that affect the sustainability of such a wide audience distribution service. As a consequence, such dispensers are not compatible with an economical model that makes possible the distribution of hand sanitizing gel at a wide scale while optimizing issues about user experience, replenishment and maintenance all together.

The purpose of the invention is to set forth a new connected dispenser in order to cope at least partially with the issues above and to provide new advantages in addition.

Another purpose of the invention is to find and use such a dispenser more easily.

Another purpose of the invention is to make the dispensing of cleaning and/or sanitizing solution easier while impeding the propagation of contaminants between two successive users of such a dispenser.

Another purpose of the invention is to make the maintenance or replenishment of such a dispenser easier.

### DISCLOSURE OF THE INVENTION

According to a first aspect of the invention, at least one of the goals above is obtained with a dispenser of a cleaning and/or sanitizing solution, wherein the dispenser includes (i) a tank designed to contain the cleaning and/or sanitizing solution, (ii) a delivery system of the cleaning and/or sanitizing solution, wherein the delivery system includes a delivery head in fluidic communication with the tank through a delivery pipe, a pump designed to regulate the flow of the cleaning and/or sanitizing solution through the delivery pipe, (iii) a command module designed to selectively monitor the pump and the dispensing head of the delivery system, (iv) a radiofrequency communication module designed for a wireless two-way communication between the dispenser and a portable communication device and to activate or deactivate the dispenser according to a command signal from the communication appliance to the radiofrequency communication module, and (v) a power source connected to the delivery system, to the command module and to the radiofrequency communication module.

The dispenser according to the first aspect of the invention can take any shape. In particular, it can include fixing members to fix it in a permanent or movable way to a vertical mount. Alternatively, the dispenser can take the form of a totem and include a baseplate to stand on the ground and remain there standing.

The dispenser is delineated by a housing that is an outer shell with any shape according to preferential uses, with all or a part of the components inside, including notably the radiofrequency communication module and/or the command module and/or the delivery system and/or the tank and/or the power source.

Preferably but not restrictively, the cleaning and/or sanitizing solution released by the dispenser according to the first aspect of the invention is a hand sanitizing solution or a solution including a soap. In addition, the cleaning and/or sanitizing solution is released by doses of a preset volume as a liquid, a gel, a foam or a nebulization, for example.

The tank has the shape of a closed container, designed to contain a preset volume of cleaning and/or sanitizing solution. The volume of the tank is preferably between 0.5 liter and 10 liters. Advantageously, the volume of the tank is between 1 liter and 5 liters to optimize both the cluttering and the number of doses of cleaning and/or sanitizing solution delivered by the dispenser before replenishment or replacement of the tank. The tank can be made of any material, notably of plastic or metal. According to a preferential embodiment, the tank is a container inside the dispenser and its housing. According to an alternative embodiment, the tank is totally or partially made of a part of the housing of the dispenser.

The tank is preferably hermetically closed to prevent the evaporation of the cleaning and/or sanitizing solution and to reduce the risk of ignition. Advantageously, the dispenser includes an access device to the tank to allow its replenishment and/or its replacement. According to a first embodiment, such an access device can take the shape of a removable trap door that is retractable or rotatably mounted and gives access to the part of the dispenser including the tank, in order to remove or introduce the tank into the housing of the dispenser. According to a second embodiment, such an access device can take the shape of a repositionable plug—preferably hermetically—that allow the replenishment of the tank with cleaning and/or sanitizing solution.

The delivery system makes it possible to circulate the cleaning and/or sanitizing solution from the tank to the delivery head while monitoring the flow of the cleaning and/or sanitizing solution. Preferably, in order to mitigate leaks of cleaning and/or sanitizing solution inside the dispenser, the delivery system is hermetically coupled, in particular at the tank and at the delivery head.

The regulation of the flow rate of the cleaning and/or sanitizing solution through the delivery system is made by the pump that is driven by the command module of the dispenser. The pump is at least designed to regulate the flow rate of the cleaning and/or sanitizing solution between zero and a nominal value above zero. In addition, the pump is driven by the command module so that it regulates the flow rate of cleaning and/or sanitizing solution to its nominal value for a preset duration that matches the delivery of a preset nominal volume of cleaning and/or sanitizing solution by the delivery head.

The delivery head includes at least one delivery nozzle designed to release the cleaning and/or sanitizing solution and to let a user of the dispenser retrieve said solution and apply it, for example on their hands. According to the intended uses and effects, the at least one delivery nozzle of the delivery head is designed to spray the cleaning and/or sanitizing solution in the form of droplets or as a jet.

The command module has the form of one or several PCB connected between each other and with the delivery system, the radiofrequency communication module and the power source of the dispenser. As non-limitative examples, the PCB of the command module can include integrated circuits for the treatment of data exchanged with the radiofrequency communication module and the delivery system. Alternatively or complementarily, the PCB of the command module can include integrated circuits to monitor the radiofrequency communication module, the delivery system and the power source. Integrated circuits can take the form of one or several controllers and/or one or several microprocessors and/or one or several microcontrollers and/or one or several field programmable gate arrays (FPGA) and/or one or several application specific integrated circuits (ASIC).

Complementarily, the command module can include and/or be connected and/or be designed to access a memory designed to store digital data. To access them, it shall be understood that the command module is designed to notably write and/or read from the memory. The PCB of the command module can include any kind of memory, such as RAM and/or ROM and/or cache and/or buffer and/or optical memory and/or erasable programmable read-only memory.

The radiofrequency communication module is designed to emit and receive a respectively inbound and outbound modulated radiocommunication signal.

The power source is preferably an electric power source, such as, for example, a battery inside the dispenser. As a non-limitative example, the battery can be a lithium battery. Alternatively, the power source can be an electric connection member between the dispenser and a neighboring electrical grid. This advantageous configuration makes it possible to electrically connect the dispenser according to the first aspect of the invention to the neighboring electrical grid. Advantageously, the electric connection member of the dispenser is a female connector, designed to be coupled with an analogous male connector.

Thus, the dispenser according to the first aspect of the invention makes it easier and healthier to release the cleaning and/or sanitizing solution to a user of said dispenser. Indeed, such a dispenser can be used, which means triggered, safely and with no physical contact with any part of the dispenser.

In addition, the dispenser according to the first aspect of the invention spares more the cleaning and/or sanitizing solution, because the doses of said solution are released independently from any action by the user of the dispenser. Contrarily, the doses of the cleaning and/or sanitizing solution provided by the dispenser are preset and automatically released in a controlled manner and are regulated by the delivery system of the dispenser under control of the command module.

As a consequence, such a dispenser is more reliable, more robust and more adapted to a massive installation in the public space, because its maintenance can be more rational and less frequent than with the known dispensers. In addition, the replenishment circuits of cleaning and/or sanitizing solution are easier to implement and less costly.

The dispenser according to the first aspect of the invention advantageously includes at least one of the following refinements, wherein the technical characteristics of these refinements can be adopted alone or in combination:
according to a first embodiment, the radiofrequency communication module includes at least one communication antenna electrically and operationally connected to the command module of the dispenser according to the first aspect of the invention. In this case, the command module is designed to respectively modulate and demodulate the inbound and outbound communication signal.
according to a second alternative or complementary embodiment, the radiofrequency communication module includes at least one communication antenna and a modulation-demodulation unit that is electrically and operationally connected to the at least one communication antenna. The modulation-demodulation unit is designed to respectively modulate and demodulate the inbound and outbound communication signal. The modulation-demodulation unit advantageously includes one or several controllers and/or one or several microprocessors and/or one or several microcontrollers and/or one or several field programmable gate arrays (FPGA) and/or one or several application specific integrated circuits (ASIC). The modulation-demodulation unit preferably includes a memory that is electrically and operationally connected to the antenna. The memory can be of any kind, for example a RAM and/or a ROM and/or a cache and/or a buffer and/or an optical memory and/or an erasable programmable read-only memory;
in either embodiment, the communication antenna is a Bluetooth antenna. Thus, the communication antenna is designed to emit and/or receive a very high frequency radio signal between 2.4 and 2.485 GHz as defined in the standard IEEE802.15.1. Possibly, the communication antenna is a low energy Bluetooth antenna (BLE);
the radiofrequency communication module includes a near field communication terminal (NFC) connected to the command module. In this case, the communication terminal is a NFC antenna that makes it possible to create a short range high frequency wireless communication, typically at 13.56 MHz. The NFC technology is described in particular in the ISO/CEI 14443 standard;
the communication antenna is a near field antenna;
the delivery system includes a sensor designed to measure a volume of cleaning and/or sanitizing solution inside the tank. Thus, the sensor makes it possible to determine whether the residual volume of cleaning and/or sanitizing solution falls under a threshold value that requires planning a maintenance or replenishment operation of the dispenser;
the command module incudes a memory, and the sensor is connected to the command module in order to store a value if the measured volume in memory.
the dispenser according to the first aspect of the invention includes a proximity sensor that is electrically connected to the command module, wherein the proximity sensor is designed to activate the delivery system when a user of the dispenser moves their hands close to said dispenser. As non-limitative examples, the proximity sensor can be an infrared sensor, an ultrasound sensor, a capacitive sensor. This advantageous design is able to detect when the user of the dispenser is in close proximity of it—particularly in close proximity of the delivery head—and it is possible to deliver the requested dose of cleaning and/or sanitizing solution.
the dispenser according to the first aspect of the invention includes at least visual communication devices to explain how it works and/or for sanitary awareness programs and/or to carry advertising. As non-limitative and complementary examples, the visual communication devices include at least one pictogram that is sticked or etched on the dispenser and/or an integrated screen for interactive communication with users. In this embodiment, the screen is preferably a liquid crystal display (LCD) or a light-emitting diode screen (LED) or an organic light-emitting diode screen (OLED), for example.

According to a second aspect of the invention, it is provided a process to control a dispenser according to the first aspect of the invention or according to any one of its refinements thanks to a portable communication device, wherein the control process includes (i) a step of identification of the portable communication device by the dispenser by transmitting an identifier of the portable communication device and reception by the radiofrequency communication module of the dispenser, and (ii) a step of release of a dose of the cleaning and/or sanitizing solution by the dispenser.

Thus, the control process of the dispenser makes it possible to command the release of a dose of the cleaning and/or sanitizing solution by the dispenser thanks to a portable communication device. The portable distribution device is preferably equipped with a custom mobile application that gives access—in addition to the unlocking of the dispensers—to services and information of public utility relative to collective sanitary strategies. More specifically, such a portable communication device, when located close to a dispenser according to the first aspect of the invention, is able to transmit wirelessly its identifier to the dispenser.

By «proximity», it is understood that whoever carries the portable communication device is located at such a distance from the dispenser that it is possible for said user to use the dispenser immediately or in seconds. In other words, by «proximity», it is understood relative to the communication capacities of the dispenser, on one hand, and of the portable communication device, on the other hand: the communication device is supposed to be located at proximity of the dispenser when the distance between them is between less than 1 cm and 50 m.

In terms of the invention, and as a non-limitative example, the identifier can be an indexer that identifies the portable communication device or a variable including an authorization of service for the portable communication device. In a first example, the indexer constituting the identifier that is received by the dispenser can take the form of an alphanumerical code that refers to the portable communication device, such as, for example, a MAC address or an identifier of the application made of a series of figures and/or letters and/or special characters. In this case, it is a unique identifier. Possibly, the identifier can be a generic identifier shared by several users, wherein the identifier guarantees that the user of the portable communication device is a subscriber of a service of use of the dispensers according to the first aspect of the invention. In the second example, the identifier can take the form of a Boolean value specifying whether the authorization of service is granted or not.

When the dispenser has received the identifier associated with the portable communication device, the identifier is compared—at a comparison step—with a reference value to determine whether the delivery step is completed or not. If the identifier from the portable communication device is acknowledged as valid and allowing the release of a dose of cleaning and/or sanitizing solution, then the dispenser releases a preset volume of said solution, which constitutes said dose. To the contrary, if the identifier is acknowledged as invalid, then the delivery step is not completed.

As a non-limitative example, a user of the dispenser who would not have enough credits to use it would transmit an invalid identifier to the dispenser.

As previously specified, the delivery step makes it possible to release a preset dose of cleaning and/or sanitizing solution to the user who requested it through their portable communication device. Thus, the dispensing step includes a step of actuation of the pump of the delivery system for a preset duration to circulate a preset volume of cleaning and/or sanitizing solution through the delivery system to the delivery head of the dispenser. When the preset duration is completed, the pump is deactivated to disrupt the flow of cleaning and/or sanitizing solution from the delivery head.

Of course, in the terms of the invention, a portable communication device is any connected object including wireless communication means in interaction with other connected objects, notably with dispensers according to the first aspect of the invention. As non-limitative examples, such a portable communication device can be a cellphone, a connected watch or any accessory connected to a cellphone, a tablet, a laptop . . . .

Thus, the control process according to the second aspect of the invention makes it possible to monitor a dispenser of cleaning and/or sanitizing solution remotely and without any physical interaction with it. Thus, it is possible to request a dose of cleaning and/or sanitizing solution without touching any interface of the dispenser. Thus, the control process makes it possible to mitigate the propagation of viruses and contaminants in public and private places, where such dispensers would stand.

The control process according to the second aspect of the invention advantageously includes at least one of the following refinements, wherein the technical characteristics of these refinements can be adopted alone or in combination:
according to a first embodiment, the identification step includes setting up a Bluetooth link between the portable communication device and the radiofrequency communication module of the dispenser. Under Bluetooth link, it is understood setting up a very high frequency radio signal between 2.4 and 2.485 GHz as defined in the standard IEEE802.15.1. Possibly, the connection is a low energy Bluetooth antenna (BLE). This first embodiment advantageously sets up a two-way communication at wider range, typically less than 100 m. According to a second embodiment, the identification step includes setting up a near field link between the portable communication device and the radiofrequency communication module of the dispenser. Under near field link, it is understood setting up a short range high frequency wireless communication—typically 13.56 MHz—as described in the standard ISO/CEI 14443 for the NFC technology (Near Field Communication). This second embodiment creates a more secure link between the dispenser and the portable communication device.
the delivery step include a step of transmission of a delivery command by the portable communication device to the dispenser through its radiofrequency communication module. Possibly, the identifier transmitted at the identification step includes simultaneously the delivery command for the cleaning and/or sanitizing solution. In this case, the transmission of the delivery command is completed simultaneously with the identification step. Alternatively, the transmission of the delivery command is completed successively with the identification step. In this case, the identifier transmitted at the identification step allows the dispenser to check at least the validity of the identifier associated with the portable communication device. Hereafter, if the identifier is considered as valid by the dispenser, then the delivery command from the portable communication device initiates the distribution step;
according to a first embodiment, the identification step of the portable communication device and the transmission step of the delivery command are made according to identical communication protocols. This design advantageously makes it easier the development of dispensers according to the first aspect of the invention and reduces their cost. In this first embodiment, the connection between the dispenser and the portable communication device is preferably a Bluetooth link for the identification step and the transmission of the delivery command. In this case, the control process according to the second aspect of the invention advantageously includes a pairing step between the dispenser and the portable communication device before the delivery step. The pairing step makes it possible to the portable communication device to reserve the use of the dispenser according to the first aspect of the invention. After this pairing, the connection between the portable communication device and the dispenser is two-way. When a first portable communication device is paired with the dispenser, it is not possible for a second portable communication device to be simultaneously paired with the same dispenser;
according to a second embodiment alternative to the first embodiment, the identification step of the portable communication device and the transmission step of the delivery command are made according to different communication protocols. This design allows a better management of the various users of a same dispenser. According to a particularly advantageous embodiment of the invention according to the second aspect, the identification of the portable communication device is a Bluetooth link and the transmission step of the delivery command is a near field link. The Bluetooth link creates a first remote link between the dispenser and the portable communication device in order to permit the identification of said device on said dispenser. To the contrary, the near field link more securely activates the dispenser thanks to the identification of the portable communication device:
the dispensing step includes a step of actuation of the pump of the delivery system for a preset duration to deliver a preset volume of cleaning and/or sanitizing solution. In particular, a volume of cleaning and/or sanitizing solution dispensed during the delivery step and corresponding to a dose of cleaning and/or sanitizing solution is between 0.5 ml and 2 ml. Preferably, the volume of a dose of cleaning and/or sanitizing solution is equal or substantially equal to 1 ml;
the control process according to the second aspect of the invention includes a location step of the dispenser relative to the portable communication device. This location step makes the access of the dispensers easier;
according to a first embodiment, according to a first embodiment, the location step includes (i) a step of reception of a geo-positioning signal by the portable communication device, (ii) an identification step of the geographic coordinates of the portable communication device, and (iii) a display step of at least one dispenser—called activatable dispensers—located within a preset perimeter from the geographical coordinates of the portable communication device.
for this first embodiment, in the location step, the geographical coordinates of one of the activatable dispensers are transmitted to the portable communication device through a wireless communication network from a central server. In this variant of the first embodiment, the geographical coordinates of the dispensers are previously input and centrally stored in the central server. Thus, a place for all dispensers of a given network of dispensers is known and accessible from the portable communication device. Alternatively, for this first embodiment, in the location step, each dispenser transmits its geographical coordinates to the portable communication device through its radiofrequency communication module. In this second variant of the first embodiment, each dispenser transmits its geographical coordinates to make it easier to locate for a communication device located in its broadcast perimeter. Thus, the dispenser emits a carrier signal of its geographical coordinates that can be received by any portable communication device located in this perimeter, which will be able to calculate its position relative to the dispenser;
the location step includes an emission step of a dispenser identifier by the dispenser through its radiofrequency communication module. This step makes it possible to better identify the dispenser. This design is particularly advantageous in the context of a high density network of dispensers, such as, for example, in some public places like railway or tube stations;
according to a particularly smart variant, the identifier transmitted by the dispenser also includes geolocation information for the dispenser—for example its geographical coordinates. In this case, the location step is included in the transmission of the identifier of the dispenser.
advantageously, the dispenser completes the emission step of the dispenser identifier at a preset frequency. This design reduces the electrical consumption of the dispenser. As a non-limitative example, the emission frequency of the identifier is between 0.1 Hz and 10 Hz. Preferably, the emission frequency of the identifier is equal or substantially equal to 1 Hz;
the control process according to the second aspect of the invention includes a transmission step of a validation status of the delivered dose of the cleaning and/or sanitizing solution to the portable communication device and through the radiofrequency communication module of the dispenser. This step makes it possible to check whether the service has been completed by the dispenser, which means that the dose of cleaning and/or sanitizing solution has been delivered to the user of the dispenser;
after the portable communication device receives the status of validation, the control process includes a step of update of the user data associated with the identifier of said portable communication device, including notably a number of uses of the dispenser.

There are various embodiments of the invention that integrate the different optional characteristics specified here according to their possible combinations.

### DESCRIPTION OF THE FIGURES

Other characteristics and advantages of the invention will appear thanks to the following description, on one hand, and thanks to several embodiments produced as a guide and non-limitative with a reference to the appended schematic pictures, on the other hand, where:

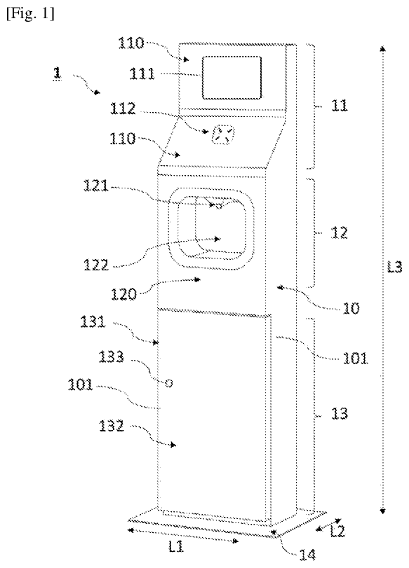

The FIG. 1 shows an embodiment of a dispenser according to the first aspect of the invention;

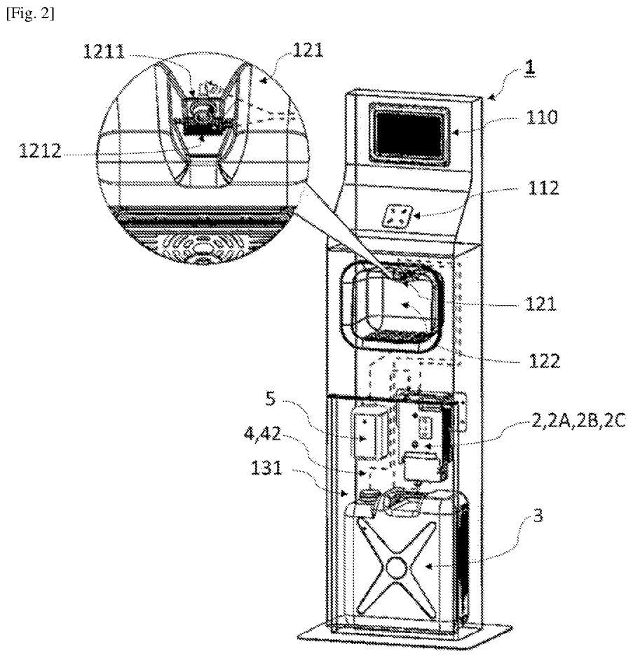

The FIG. 2 shows a cutaway view of the dispenser of FIG. 1;

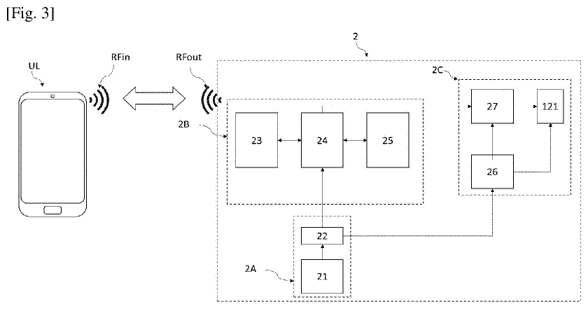

The FIG. 3 shows a schematical view of the electronic design of the dispenser according to the first aspect of the invention;

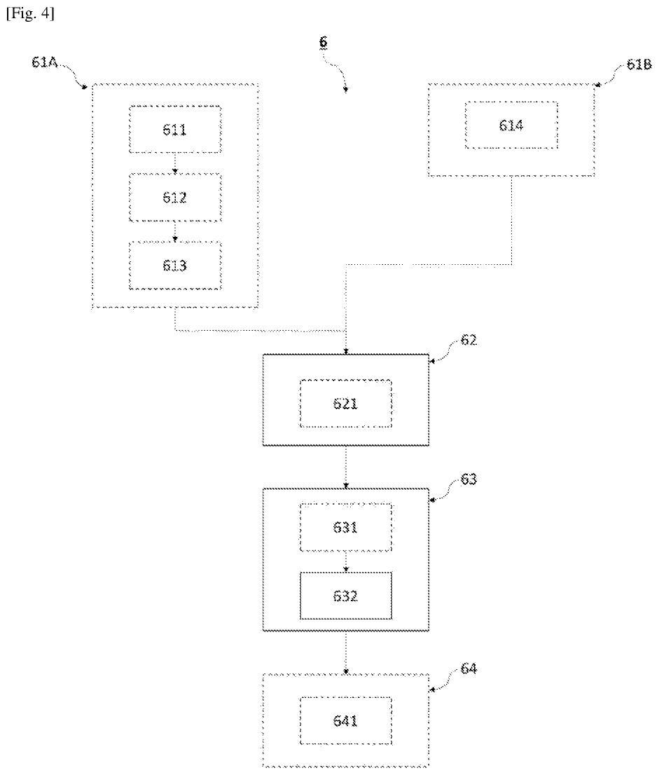

The FIG. 4 shows a synoptic view of the control process according to the second aspect of the invention.

Of course, the characteristics, the variants and the various embodiments of the invention can be associated under various combinations, as far as they are not mutually incompatible or exclusive. You could conceive variants of the invention, which only include a selection of characteristics as described below, apart from the other describe characteristics, as far as this selection of characteristics is enough to give a technical advantage or to distinguish the invention from the previous state of the art.

In particular, all described variants and embodiments can be associated if it is technically possible.

On the figures, common items to several figures keep the same reference.

### DETAILED DESCRIPTION OF THE INVENTION

The FIGS. 1, 2, and 3, describe a dispenser 1 according to the first aspect of the invention that dispense a preset quantity of cleaning and sanitizing solution in a controlled manner. In a particularly advantageous manner, the dispenser 1 is remote controlled by a portable communication device not visible on the FIGS. 1 and 2. The FIGS. 1 and 2 show a three-dimensional view of an embodiment of a dispenser 1 according to the invention, and the [FIG. 3] shows a schematic view of an electronic system 2 of said dispenser 1.

The dispenser 1 can include fixing members to fix it to a vertical mount, or the dispenser 1 can take the form of a totem, such as visible in the implementation illustrated in the FIGS. 1 and 2, where the dispenser 1 includes a base plate 14 to stand on the ground and remain there standing.

In the embodiment in the FIGS. 1 et 2, the dispenser 1 is delineated by a housing 10 that is an outer shell with all or a part of the components of the dispenser 1 inside. In particular, the housing 10 includes at least the following components of the dispenser 1.
a tank 3 designed to contain the cleaning and/or sanitizing solution;
a delivery system 4 of the cleaning and/or sanitizing solution;
an electronic system 2 designed to monitor the functioning of the dispenser 1, wherein the electronic system 2 includes a command module 2C designed to monitor the delivery system 4, a radiofrequency communication module 2B designed for a wireless two-way communication between the dispenser 1 and a portable communication device, an electrical power module 2A of the dispenser 1.

In the embodiment of the FIGS. 1 and 2, the housing 10 of the dispenser 1 has a longitudinal vertical shape and includes an upper part 11, a lower part 13 and a middle part 12. The lower part 13 of the housing 10 of the dispenser 1 has a rectangular or cylindrical shape. The middle part 12 of the housing 10 is located in line with the lower part 13. The middle part 12 of the housing 10 has a rectangular or cylindrical shape.

To narrow the hold on the ground of the dispenser 10, a first lateral dimension L1 of the housing 10 is between 400 mm and 600 mm Preferably, the first lateral dimension L1 of the housing 10 is 550 mm. The first lateral dimension L1 is measured between two lateral faces 101 of the carter 10. A second lateral dimension L2 of the housing 10 is between 300 mm and 500 mm Preferably, the second lateral dimension L2 of the housing 10 is 400 mm A height L3 of the dispenser 1 is advantageously between 1200 mm and 1800 mm to make its user easier for a standing user in front of it. Preferably, a total height of the dispenser 1 is 1600 mm.

The housing 10 of the dispenser 1 can be made of any material that makes it compatible for integration in the public landscape and resistant to intensive use and/or to the weather. As non-limitative examples, the housing 10 can be totally or partially made of plastic or metallic materials.

In the embodiment shown on the [FIG. 2], the lower part 13 of the housing 10 of the dispenser includes the tank 3 and the electronic system 2 inside a specific housing 131. A removable or rotatably mounted trap door 132 gives access to the receptacle 131 of the lower part 13 of the housing 10, notably for maintenance and/or replenishment of the tank 3. The trap door is closed by a locking member 133, such as a lock.

The middle part 12 of the housing 10 of the dispenser 1 includes a recess 122 where a user of the dispenser 1 can put his hands to receive the dose of cleaning and/or sanitizing solution. The recess is advantageously located at a height between 1000 mm and 1300 mm above the ground and/or above the base plate 14.

In the upper part 11, the housing 10 incudes a bevel part: a front side 110 of the housing 10 is tilted so that the second lateral dimension L2 of the housing 10 decreases when closer to an upper vertical end of the dispenser 1. At last, the upper part 11 of the dispenser 1 includes a visual communication device 111 to explain how it works and/or for sanitary awareness programs and/or to carry advertising. In the embodiment of the FIGS. 1 and 2, the visual communication device has the shape of an integrated screen for interactive communication with the users. The screen is preferably a liquid crystal display (LCD) or a light-emitting diode screen (LED) or an organic light-emitting diode screen (OLED), for example.

The delivery system 4 of the dispenser 1 makes it possible to deliver a preset dose of the cleaning and/or sanitizing solution to a user of the dispenser 1. The delivery system 4 includes a delivery head 121 in fluidic communication with the tank 3 through at least one delivery pipe 42, and a pump designed to regulate the flow of the cleaning and/or sanitizing solution through the delivery pipe.

The delivery pipes 42 establish a fluidic communication between the tank 3 an the delivery head 121 to send the cleaning and/or sanitizing solution to the delivery head 121. Advantageously, the delivery pipes 42 of the delivery system 42 are hermetically coupled to the tank 3 and to the delivery head 121.

The tank 3 has the shape of a closed container, designed to contain a preset volume of cleaning and/or sanitizing solution. The volume of the tank is preferably between 0.5 liter and 10 liters. The tank is preferably located in the lower part 13 of the dispenser 1 to help it stand and for an easier design. The tank 3 is made of plastic or metal. Additionally, the tank 3 is hermetically closed to prevent the evaporation of the cleaning and/or sanitizing solution and to reduce the risk of ignition.

The delivery system 4 makes it possible to circulate the cleaning and/or sanitizing solution from the tank 3 to the delivery head 121 while monitoring the flow of the cleaning and/or sanitizing solution. The regulation of the flow rate of the cleaning and/or sanitizing solution through the delivery system 4 is made by the pump that is driven by the command module 2C of the dispenser 1.

The pump of the delivery system 4 is driven by the command module 2C of the electronic system 2 so that it regulates the flow rate of cleaning and/or sanitizing solution to a non-zero nominal value for a preset duration that matches the delivery of a preset nominal volume of cleaning and/or sanitizing solution by the delivery head 121. When the nominal volume is reached, the command module 2C drives the pump to disrupt the flow of cleaning and/or sanitizing solution through the delivery head 121.

The delivery head 121 of the delivery system 4 is located at the recess 122 of the middle part 12 of the housing 10. More precisely, in the embodiment of the FIGS. 1 and 2, the delivery head is located at an upper wall of the recess 121, so that the cleaning and/or sanitizing solution can fall into the recess simply by gravity. Notably, this advantageously design makes it possible to recover in a collecting bin 5 any excess of cleaning and/or sanitizing solution that would not be used by the user of the dispenser 1. To do so, the recess 122 includes, at its lower wall face to the delivery head 121, a drainage grid and a collecting pipe with a fluidic coupling to the collecting bin.

The delivery head 121 includes at least one delivery nozzle 1211 designed to release the cleaning and/or sanitizing solution and to let a user of the dispenser 1 retrieve said solution and apply it, for example on their hands. According to the intended uses and effects, the at least one delivery nozzle 1211 of the delivery head 121 is designed to spray the cleaning and/or sanitizing solution in the form of droplets or as a jet.

Possibly, the delivery system 4 also includes a sensor designed to measure a volume of cleaning and/or sanitizing solution inside the tank 3. Thus, the sensor makes it possible to determine whether the residual volume of cleaning and/or sanitizing solution falls under a threshold value that requires planning a maintenance or replenishment operation of the dispenser 1;

The delivery system 4 can also include a proximity sensor 1212 that is electrically connected to the command module 2C, wherein the proximity sensor 1212 is designed to activate the delivery system 4 when a user of the dispenser 1 moves their hands close to said dispenser 1, in particular close to its delivery head 121. As non-limitative examples, the proximity sensor can be an infrared sensor, an ultrasound sensor, a capacitive sensor.

Preferably but not restrictively, the cleaning and/or sanitizing solution released by the dispenser 1 according to the first aspect of the invention is a hand sanitizing solution or a solution containing a soap. In addition, the delivery head 121 releases the cleaning and/or sanitizing solution by doses of a preset volume as a liquid, a gel, a foam or a nebulization, for example.

In reference to [FIG. 3], here is the electronic system 2 of the dispenser 1 according to the first aspect of the invention described with more details. The electronic system includes the electrical power module 2A, the radiofrequency communication module 2B and the command module 2C.

The electrical power module 2A is designed to produce the electrical energy necessary to the functioning of the radiofrequency communication module 2B and the command module 2C through a power source 21. According to a first embodiment, the electrical power module 2A includes a power source that allows the dispenser 1 to work autonomously. The power source takes the shape of a rechargeable or non-rechargeable battery. Alternatively, the electrical power module takes the shape of an electric connection member between the dispenser 1 and a neighboring electrical grid. In this embodiment, the dispenser does not include any power source and the electrical connection member makes it possible to easily connect the dispenser 1 with the neighboring electrical grid. As a non-limitative example, the electrical connection member of the dispenser 1 is a power outlet.

Thus, the electrical power module 2A is made of the power source 21 and a conditioning device 22.

Thus, the power source 21 can generate an electric power signal that drives the other modules 2B, 2C of the electronic system 2; and the conditioning device makes it possible to adjust the electric characteristics of the electric power signal to the requirements of each module 2B, 2C. As a non-limitative example, the conditioning device makes it possible to selectively generate a tension and/or a current and/or a frequency and/or a phase of the electric power signal. The conditioning device 21 is designed to distinctly generate a first electric power signal for the radiofrequency communication module 2B and a second electric power signal for the command module 2C.

The command module 2C is designed to monitor the various members of the dispenser 1, such as, notably, the delivery system 4 and/or the proximity sensor 1212 and/or the screen that is the visual communication device 111. In particular, the command module 2C includes:
a monitoring device 27 of the proximity sensor 1212, designed to focus the proximity sensor to detect a user who put their hands in to the recess 122 of the dispenser 1; and
a controller 26 designed to monitor the pump and the delivery head 121 of the delivery system 4, wherein the controller is designed to electrically power the pump and/or the delivery head 121 when the proximity sensor 1212 has detected a user, who has put their hands into the recess 122 of the dispenser 1.

The radiofrequency communication module 2B is designed to emit and receive a respectively inbound RFin and outbound RFout modulated radiocommunication signal. The outbound modulated communication signal RFout is generated by the radiofrequency communication module 2B and the inbound modulated communication signal RFin is generated by a portable communication device UL located close to the dispenser 1 and detected by said radiofrequency communication module 2B.

To do so, the radiofrequency communication module 2B includes:
a treatment unit 24 notably designed to modulate the outbound modulated communication signal RFout and demodulate the inbound modulated communication signal RFin according to a plurality of communication protocols; and/or
a communication antenna 23, 25 designed to emit and/or receive a modulate radiofrequency signal.

The dispenser 1 according to the first aspect of the invention is designed to communicate according to various protocols. Preferably, it is designed only for Bluetooth communication or only for near field communication or selectively for Bluetooth or near field communication. Several communication modes between the dispenser 1—through its radiofrequency communication module 2B—and the portable communication device UL will be described below with reference to [FIG. 4].

In the electronic system 2 as in [FIG. 3], the radiofrequency communication module 2B includes a Bluetooth antenna 23 and a NFC antenna 25, wherein the treatment unit 24 makes it possible to communicate selectively according to either one of the communication protocols according to the intended uses. This way, the treatment unit 24 constitutes both a modulation-demodulation unit that is electrically and functionally connected to the Bluetooth antenna 23 and a communication terminal that is electrically and functionally connected to the NFC antenna 25, as specified above.

To make the interaction between the portable communication device UL and the dispenser 1 easier, the Bluetooth antenna 23 and/or the NCF antenna 25 are preferably located at the upper part 11 of the dispenser 1, in an intermediary position between the visual communication device 111 and the recess 122 close to or inside a cartridge 112 on the frontal surface of the housing 10.

The treatment unit 24 of the radiofrequency communication module 2B advantageously includes one or several controllers and/or one or several microprocessors and/or one or several microcontrollers and/or one or several field programmable gate arrays (FPGA) and/or one or several application specific integrated circuits (ASIC). The treatment unit 24 also includes a memory that is electrically and operationally connected to the antenna.

The electronic system 2 of the dispenser 1 according to the invention takes the form of one or several PCB connected between each other—for example one per module 2A, 2B, 2C—and with the delivery system 4 of the dispenser 1. As non-limitative examples, the PCB of each module 2A, 2B, 2C of the electronic system 2 can include integrated circuits for the treatment of the exchanged data between the command module 2C with the radiofrequency communication module 2B and the delivery system 4 and/or for the monitoring of the radiofrequency communication module 2B, of the delivery system 4 and of the electrical power module 2A. Integrated circuits can take the form of one or several controllers and/or one or several microprocessors and/or one or several microcontrollers and/or one or several field programmable gate arrays (FPGA) and/or one or several application specific integrated circuits (ASIC).

Complementarily, each module 2A, 2B, 2C that form the electronic system 2 can include and/or be connected and/or be designed to access a memory designed to store digital data, for example a RAM and/or a ROM and/or a cache and/or a buffer and/or an optical memory and/or an erasable programmable read-only memory.

The [FIG. 4] shows a synoptic view of the control process 6 according to the second aspect of the invention. The control process 6 makes it possible to monitor the dispenser 1 as described above in any one of its embodiments thanks to a portable communication device UL to get a dose of cleaning and/or sanitizing solution. To do so, the control process 6 includes:
an identification step 62 of the portable communication device UL by the dispenser 1; and
a step of release 63 of a dose of the cleaning and/or sanitizing solution by the dispenser 1.

The identification step 62 makes it possible to the dispenser 1 to receive—through its radiofrequency communication module 2B—a, identifier transmitted from the portable communication device UL located nearby.

The concept of proximity depends on the communication protocol that the dispenser 1 is using. As a rule, a portable communication device UL is considered as being close to the dispenser 1 if the dispenser can communicate with it through one of its communication protocols, which means, preferably, through a Bluetooth or a NFC link. For illustrative purpose, in the case of a Bluetooth link between the dispenser 1 and the portable communication device UL, such a link can be established if said portable communication device UL is located within ca. 50 m from the dispenser 1. In the case of a NFC link between the dispenser 1 and the portable communication device UL, such a link can be established if said portable communication device UL is located within ca. 10 cm from the dispenser 1.
During the identification step 62, the control process 6 includes a connection step 621 between the dispenser 1 and the portable communication device UL. According to a first embodiment, during the connection step 621, the dispenser 1 sets up a link with the portable communication device UL—in particular in the case of a Bluetooth link. According to a second embodiment of the connection step 621, the portable communication device UL sets up a link with the dispenser 1—in particular in the case of a NFC link.

Thus, contrarily to the known dispensers, the dispenser 1 according to the invention only works if an identifier has been recognized by said dispenser 1. So, these dispensers cannot be used by anyone, but they are locked by an application that unlocks dispensers if some conditions are met.

Preferably, the identification step 621 sets up a two-way link between the dispenser 1 and the portable communication device UL. Then, the portable communication device UL sends to the dispenser 1 at least one parameter that the dispenser 1 recognizes to launch the release of cleaning and/or sanitizing solution. For illustrative purpose, the parameters can be a unique identifier of the portable communication device UL, a Boolean value, an activation command . . . .

The step of release 63 includes a step of actuation 632 of the delivery system 4 of the dispenser 1 to deliver the expected dose of cleaning and/or sanitizing solution. Possibly, the step of release 63 includes a prior step of transmission 631 of a dispensing command by the portable communication device UL to the dispenser 1 through its radiofrequency communication module 2B. In this case, the identifier transmitted at the identification step 62 allows the dispenser to check at least the validity of the identifier associated with the portable communication device UL. Hereafter, if the identifier is considered as valid by the dispenser 1, then the delivery command from the portable communication device UL initiates the distribution step 1, more specifically the step of actuation 632 of the delivery system 4 of the dispenser 1.

Advantageously, the identification step 62 of the portable communication device UL by the dispenser 1 is made by a Bluetooth link, while the transmission step 631 of the dispensing command is made by a NFC link between the portable communication device UL and the dispenser 1.

Alternatively, the identification step of the portable communication device UL by the dispenser 1 and the transmission step 631 of the dispensing command are made by a Bluetooth link.

Alternatively again, the identification step of the portable communication device UL by the dispenser 1 and the transmission step 631 of the dispensing command are made by a NFC link.

As seen on the [FIG. 4], the control process 6 according to the second aspect of the invention can include a prior location step 61A, 61B of the dispenser 1 relative to the portable communication device UL. This location step makes the access of the dispensers 1 easier to the users, in particular in urban environment.

According to a first embodiment, the location step 61A includes:
a step of reception 611 of a geo-positioning signal by the portable communication device UL;
an identification step 612 of the geographic coordinates of the portable communication device UL; and
a display step 613 of at least one dispenser 1—called activatable dispensers—located within a preset perimeter from the geographical coordinates of the portable communication device UL.

In this first embodiment, the geographical coordinates of all dispensers 1 are preset and those relative to the activatable dispensers 1 are then transmitted to the portable communication device UL through a wireless communication network from a central server. The geographical coordinates of the dispensers 1 are previously input and centrally stored in the central server. This embodiment makes it possible to know the place of any dispenser 1, no matter the position of the portable communication device UL or of the dispensers 1.

Alternatively, for this first embodiment, in the location step 61, each dispenser 1 transmits its geographical coordinates to the portable communication device UL through its radiofrequency communication module 2B. Thus, the dispenser 1 emits a carrier signal—for example through its Bluetooth antenna 23—of its geographical coordinates that can be received by any close portable communication device located UL, which will be able to calculate its position relative to the dispenser 1.

According to a second embodiment, the location step 61B includes an emission step 614 of a dispenser identifier by the dispenser 1 through its radiofrequency communication module 2B. In this embodiment, a central server keeping all geographical coordinates of the dispensers 1 is not needed, since each dispenser transmits data including its location information.

Advantageously, the dispenser 1 completes the emission step 614 of the dispenser identifier at a preset frequency.

The emission step 614 is completed in a non-specific manner according to a “broadcast” communication mode:

all portable communication devices within the range of the dispenser 1 can thus receive and detect the transmitted dispenser identifier. As a non-limitative example, the emission step 614 is periodically completed at a frequency equal or substantially equal to 1 Hz.

As a rule, the location step 61 is completed through a Bluetooth-type communication via the radiofrequency communication module 2B of the dispenser 1.

At last, the control process 6 can also include a transmission step 64 of a validation step for the delivered dose of cleaning and/or sanitizing solution to the portable communication device UL and through the radiofrequency communication module 2B of the dispenser 1. This step makes it possible to check whether the service has been completed by the dispenser 1, which means that the dose of cleaning and/or sanitizing solution has been delivered to the user of the dispenser 1. Afterward, the transmission step 64 advantageously includes an update step 641 of the user data associated to the identifier of said portable communication device UL, including notably a number of uses of the dispenser. This step makes it possible to synchronize an application on the portable communication device UL that monitors the dispensers 1 with the use of said dispensers 1 by people carrying said portable communication device UL. Of course, the update step 641 is after the portable communication device has received the status of validation from the portable communication device UL.

To sum up, the invention deals with a dispenser 1 of a cleaning and/or sanitizing solution with a radiofrequency communication module 2B for remote monitoring by a portable communication device UL to deliver a preset dose of the cleaning and/or sanitizing solution. The invention also deals with a control process 6 of such a dispenser 1, wherein the control process 6 includes an identification step 62 of the portable communication device UL by the dispenser 1 and—under condition of authorization after identification—a step of release 63 of the cleaning and/or sanitizing solution.

Of course, the invention is not restricted to the examples above, and many refinements may be added to these example in the framework of the invention. In particular, the other characteristics, shapes, variants and embodiments of the invention can be associated with one another under various combinations, as far as they are not mutually incompatible or exclusive. In particular, all variants and embodiments above can be associated.

1. A dispenser for a cleaning and/or sanitizing solution, wherein the dispenser includes:
a tank designed to contain the cleaning and/or sanitizing solution;
a delivery system of the cleaning and/or sanitizing solution, wherein the delivery system includes a delivery head in fluidic communication with the tank through a delivery pipe, a pump designed to regulate the flow of the cleaning and/or sanitizing solution through the delivery pipe;
a command module designed to selectively monitor the pump and the delivery head of the delivery system;
a radiofrequency communication module designed for a wireless two-way communication between the dispenser and a portable communication device, and to activate and deactivate the dispenser according to a control signal from the communication appliance to the radiofrequency communication module;
a power source connected to the delivery system, to the command module and to the radiofrequency communication module.

2. A dispenser according claim 1, wherein the radiofrequency communication module includes at least a communication antenna and a modulation-demodulation unit-connected to at least one communication antenna.

3. A dispenser according to the previous claim, wherein the communication antenna is of a Bluetooth antenna.

4. A dispenser according to claim 2, wherein the communication antenna is a near field communication antenna.

5. Control process of a dispenser according to claim 1 thanks to a portable communication device, (UL), wherein the control process includes the following steps:
an identification step of the portable communication device by the dispenser thanks to the transmission of an identifier of the portable communication device (UL) that is received by the radiofrequency communication module of the dispenser;
a step of release of a dose of the cleaning and/or sanitizing solution by the dispenser.

6. Control process according to claim 5, wherein the identification step includes setting up a Bluetooth link between the portable communication device (UL) and the radiofrequency communication module of the dispenser.

7. Control process according to claim 5, wherein the identification step includes setting up a near field link between the portable communication device (UL) and the radiofrequency communication module of the dispenser.

8. Control process according to claim 5, wherein the identification step includes a transmission step of a delivery command by the portable communication device (UL) to the dispenser through its radiofrequency communication module.

9. Control process according to claim 8, wherein the identification step of the portable communication device (UL) and the transmission step of the delivery command are made according to identical communication protocols.

10. Control process according to claim 8, wherein the identification step of the portable communication device (UL) and the transmission step of the delivery command are made according to different communication protocols.

11. Control process according to claim 10, wherein the identification step of the portable communication device (UL) is a Bluetooth link and the transmission step of the delivery command is a near field link.

12. Control process according to claim 5, wherein the control process includes a location step of the dispenser relative to the portable communication device (UL).

13. Control process according to claim 12, wherein the location step (61A, 61B) includes:
a reception step of a geo-positioning signal by the portable communication device (UL);
an identification step of the geographic coordinates of the portable communication device (UL);
a display step of at least one dispenser—called activatable dispensers—located within a preset perimeter from the geographical coordinates of the portable communication device (UL).

14. Control process according to claim 12, wherein the location step includes an emission step of a dispenser identifier by the dispenser through its radiofrequency communication module.

15. Control process according to claim 15, wherein the emission step of the dispenser identifier is made at a frequency that is preset by the dispenser.
